(19) United States
(12) Patent Application Publication
(10) Pub. No.: US 2007/0048294 A1
(43) Pub. Date: Mar. 1, 2007

(54) METHOD FOR INHIBITING TELOMERASE ACTIVITY

(76) Inventor: Jong-Gu Park, Daegu (KR)

Correspondence Address: Joseph Hyosuk Kim, Ph.D.;JHK Law, P.O. Box 1078, La Canada, CA, 91012-1078, US

(21) Appl. No.: 11/468,733

(22) Filed: Aug. 30, 2006

Related U.S. Application Data
(60) Provisional application No. 60/596,090, filed on Aug. 30, 2005.

(51) Int. Cl. A61K 48/00; C07H 21/02

(52) U.S. Cl. 424/93.21; 514/44; 536/23.1

(57) ABSTRACT

The present application discloses a purified covalently closed antisense molecule, which specifically inhibits expression of human telomerase by specifically binding to nucleic acid encoding human telomerase.

### BACKGROUND OF THE INVENTION

1. Field of the Invention

The present invention relates to the field of biotechnology and especially antisense therapy using closed covalent antisense molecule that is targeted to telomerase. The invention also relates to a method of delivering the antisense molecule to a cell. The invention further relates to a method of treating diseases caused by the production and activation of telomerase, in particular tumorigenesis and cancer.

2. General Background and State of the Art

Telomeres are specialized DNA structures composed of 6 base repeat sequence (TTAGGG) at the end of each eukaryotic chromosome. This structure protects chromosomes from end to end fusion, degradation, and rearrangements, which maintain the integrity of chromosomes (Blackburn, 1991). Telomeres become progressively shortened by 50-200 bp with each cell division due to inability of DNA polymerase to replicate the end of chromosomes in somatic cells. When the end of a shortened chromosome reaches a critical point, the shortening is believed to induce senescence of cells. Cellular senescence is caused by the induction of DNA damage responses, including activation of p53 or p21 (Harley et al; 1990; Allsopp et al; 1992).

Telomerase is a ribonucleoprotein DNA polymerase that elongates telomeric repeats of telomere in eukaryotic cells and play an important role in multiple cellular processes including cell differentiation, senescence, proliferation, inhibition of apoptosis, tumorigenesis, and possibly DNA repair and drug resistance (Urquidi et al; 2000; Fu et al; 1999; Nugent et al; 1998; Ishikawa et al; 1999). Immortalized cells show activation of telomerase and thus maintaining the telomere structure of a chromosome (Kim et al; 1994). Telomerase activity is detected in the majority of malignant tumors while it is not detectable in normal human somatic cells. It indicates that telomerase activation is an important factor for neoplastic transformation. The activation of telomerase in tumor cells makes telomerase an attractive therapeutic target.

Human telomerase is composed of three major subunits: hTR (Human telomerase RNA template) (Feng et al; 1995), hTERT (Human telomerase reverse transcriptase) (Nakamura et al; 1997; Meyerson et al; 1997), and TP1 (Telomerase associated protein1) (Harrington et al; 1997; Nakayama et al; 1997). The RNA template, hTR contains the sequence of AAUCCCAAU through which telomerase can extend telomeric repeats. Among the 3 major components of human telomerase, antisense-based strategies have been attempted against hTR and hTERT in both in vitro and in vivo studies to inhibit telomerase activity. These inhibitors of hTR template have adopted antisense chemistries of 2-5 A antisense, peptide nucleic acid, and ribozymes (Mukai et al; 2000; Herbert et al; 1999; Glukhov et al; 1998; Norton et al; 1996). Antisense to hTR eliminates the RNA template for telomere synthesis. Gastric carcinoma cells treated with antisense hTR lose telomeric repeats, resulting in cell death or cellular senescence (Naka et al; 1999). Tumor cells transfected with antisense nucleic acid against hTR inhibits telomerase activity and subsequently induces either apoptosis or differentiation (Kondo et al; 1998). Inhibition of telomerase activity is likely to be very effective in limiting the growth of various kinds of cancer cells, which is an important target for the development of new therapeutics for the development of anti-neoplastic therapies (Shay and Wright, 1996; Hahn et al; 1999; Kanazawa et al; 1996).

We have developed a series of antisense molecules with enhanced stability and low toxicity (Moon et al; 2000A; Moon et al; 2000B). Among these, ribbon antisense is the latest and have been shown to have a good antisense activity with exceptional stability, natural nucleotide composition, and easy construction. Successful antisense activity is dependent on efficient cellular uptake of antisense molecules as well as improved antisense properties. When combined with cationic liposomes, antisense molecules are reported to show enhanced cellular uptake (Matsuda et al; 1996). DNA transfection mediated by cationic liposomes can be further enhanced upon forming tripartite DPL complexes containing a short peptide of the protein transduction domain (manuscript in preparation).

The present application is directed to a covalently closed antisense nucleic acid molecule targeted to hTR, which is designed and tested for effective removal of hTR RNA in several cancer cell lines that show telomerase activation. To achieve an optimal antisense activity, much enhanced uptake of the antisense nucleic acid was adopted by employing the DPL (DNA/peptide/liposome) complex for both in vitro and in vivo applications.

### SUMMARY OF THE INVENTION

In one aspect, the invention is directed to a purified covalently closed antisense molecule, which specifically inhibits expression of human telomerase by specifically binding to nucleic acid encoding human telomerase. The covalently closed antisense molecule may have at least two loops separated by a stem structure, wherein at least one loop comprises a target antisense sequence that specifically binds to nucleic acid encoding human telomerase. The molecule may specifically bind to nucleic acid encoding hTR. Further, the molecule may include a sequence, which is substantially similar to SEQ ID NO:1, SEQ ID NO:2, SEQ ID NO:3, SEQ ID NO:4, or SEQ ID NO:5.

In another aspect, the invention is directed to a method of making the above molecule, which includes ligating together at least two linear antisense molecules with stem-loop structure having either or both 5′ or 3′ ends be substantially complementary to each other so that a covalently closed antisense molecule is made. The linear antisense molecule may be specific for the same target nucleic acid or a different nucleic acid.

In yet another aspect, the invention is directed to a method of inhibiting expression of telomerase comprising contacting a sample comprising telomerase expressing cells with the above-described covalently closed antisense molecule. The invention is also directed to a method of treating a condition caused by expression of telomerase, comprising administering the above-described covalently closed antisense molecule to a subject in need thereof. The condition may be cancer. The cancer may be carcinoma, sarcoma or any other type of cancer. In particular, the cancer may be lung cancer, liver cancer, colon cancer, cervical cancer, or melanoma cancer.

In still another aspect, the invention is directed to a method for preventing proliferation of cells or reducing tumor growth or size, comprising administering a composition comprising the above-described covalently closed antisense molecule to a subject in need thereof. The tumor may be carcinoma, or any other types of tumor including sarcoma. In particular, the tumor may be lung tumor, liver tumor, colon tumor, cervical tumor, or melanoma tumor.

In another aspect, the invention is directed to a composition comprising the above-described covalently closed antisense molecule, tat or tat-like peptide, and a carrier composition. The carrier may be a liposome, and the covalently closed antisense molecule may be targeted to hTR.

In another aspect, the invention is directed to a method of delivering the above-described covalently closed antisense molecule to a cell, comprising contacting the cell with the covalently closed antisense molecule, a tat or tat-like peptide and a carrier composition. The tat or tat-like peptide and the carrier composition are mixed before contacting the cell.

In another aspect, the invention is directed to a method for treating cancer comprising administering a combination of component (i) a composition comprising the above-described molecule; and component (ii) radiation therapy, immunotherapy or chemotherapy to a subject in need thereof, with sufficient dosage or amount of the combination of component (i) and component (ii) to be effective for treating cancer or the symptoms of cancer.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1a 1b 1c show a schematic representation of ribbon antisense to human telomerase RNA. (a) The complete sequence of hTR RNA is represented by a thick horizontal solid bar. Five target sequences are denoted as 5 sub frames in the horizontal bar. hTR 13701, hTR 13702, hTR 13703, hTR 13704, and hTR 13705 are depicted as antisense sequence specific for the 5 sub frames regions. Two control oligo sequences, mismatched and scrambled oligos, are also shown in the table as hTR13706 and hTR13707. (b) A stem-loop antisense to hTR RNA is shown with the 5′ end phosphorylated. A RiAS oligo to hTR RNA containing 2 identical molecules of the hTR antisense oligo joined a stem with two loops at both ends of the molecule. (c) RiAS oligos to hTR were electrophoretically analyzed on a 12% polyacrylamide gel. Lane 1, 50 mer hTR molecules; lane 2, 100 mer RiAS oligos formed by ligation of two hTR molecules; lane 3, the ligated RiAS molecules after treatment of exonuclease I.

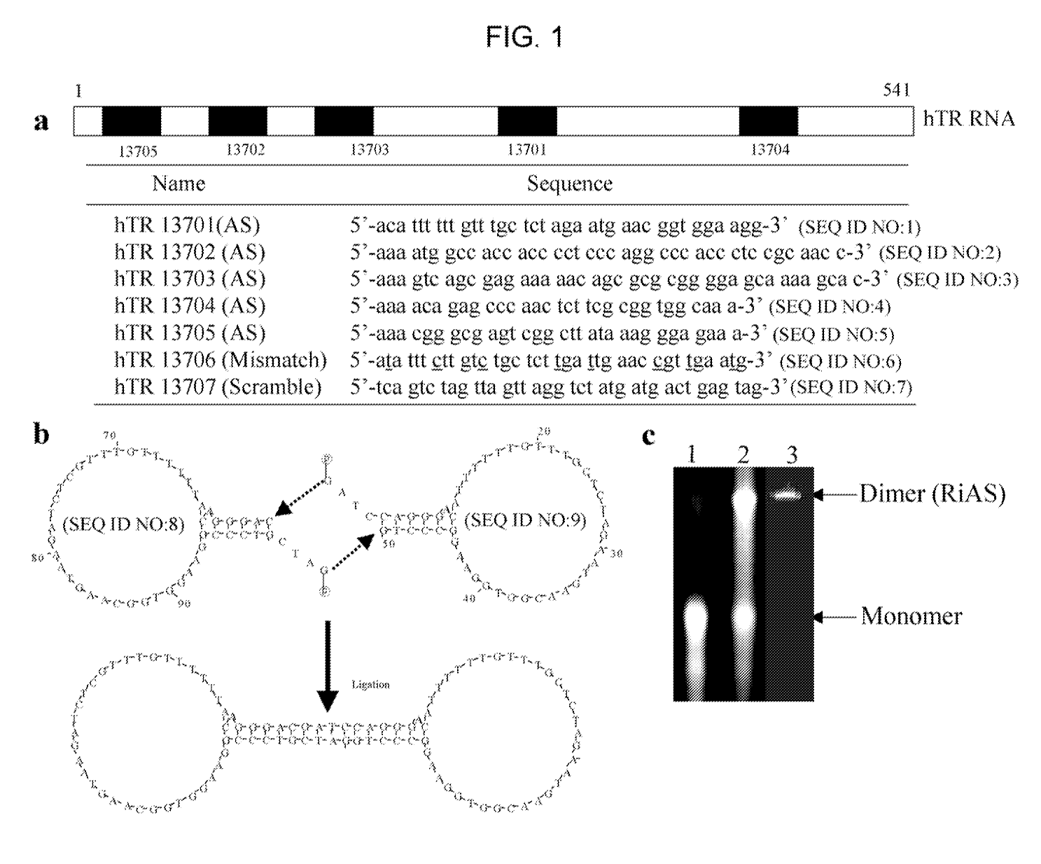

FIGS. 2a and 2b show hTR expression and specific antisense activity of ribbon antisense to hTR RNA. (a) Expression of hTR was examined in 6 cancer cell lines, lane 1; A549, lane 2; NCI H1299, lane 3; Hep3B, lane 4; SW480, lane 5; HeLa, lane 6; A 375 SM and lane M; DNA size marker of 100 bp ladder. (b) Specific reduction of hTR RNA in HeLa cells by ribbon antisense to hTR RNA. Cells were transfected with a tripartite DPL (AS oligo DNA/Tat-peptide/Lipofectamine) complex. Five different ribbon AS oligos were used and RT-PCR assays were performed. Lane M, 100 bp ladder, lane 1; sham, lane 2; liposome alone, lane 3; hTR 13701 (5′-aca ttt ttt gtt tgc tct aga atg aac ggt gga agg-3′ (SEQ ID NO:1)), lane 4; hTR 13702 (5′-aaa atg gcc acc acc cct ccc agg ccc acc ctc cgc aac c-3′ (SEQ ID NO:2)), lane 5; hTR 13703 (5′-aaa gtc agc gag aaa aac agc gcg cgg gga gca aaa gca c-3′ (SEQ ID NO:3)), lane 6; hTR 13704 (5′-aaa aca gag ccc aac tct tcg cgg tgg caa a-3′ (SEQ ID NO:4)) and 7; hTR 13705 (5′-aaa cgg gcg agt cgg ctt ata aag gga gaa a-3′ (SEQ ID NO:5)).

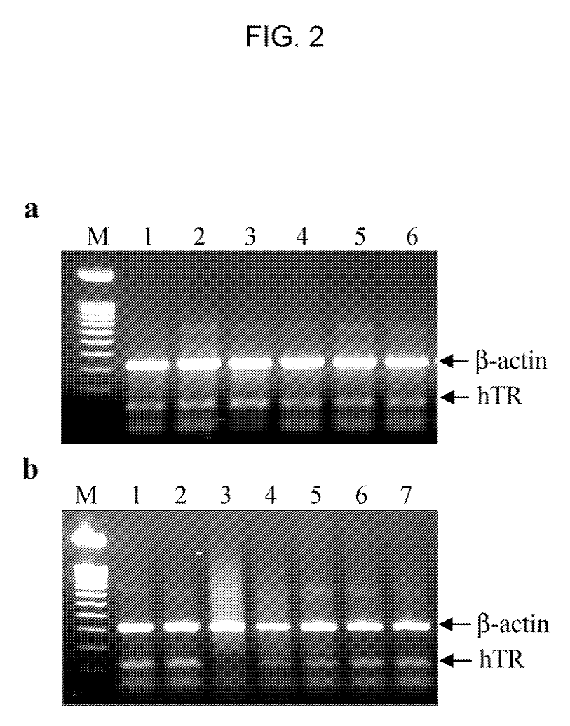

FIGS. 3a-3e show inhibition of hTR level by hTR-RiAS in various kinds of cancer cell lines. (a) Specific reduction of hTR RNA by hTR-RiAS in Colon cancer cell line (SW 480): Lane 1; 100 bp ladder, lane 2; Sham, lane 3; liposome only, lane 4; Scramble hTR, lane 5; mismatch and lane 6 hTR-RiAS. (b) Dose dependent specific reduction of hTR RNA by hTR-RiAS. HeLa cells were transfected with a tripartite lipoplex (hTR-RiAS/Tat-peptide/Lipofectamine) of different doses of hTR-RiAS (0.1 μg, 0.5 μg and 1.0 μg) and performed RT-PCR assay. Lane 1; 100 bp ladder, lane 2; sham, lane 3; liposome only, Lane 4; 0.1 μg hTR-RiAS, lane 5; 0.5 μg hTR-RiAS and lane 6; 1.0 μg hTR-RiAS. Bands shown in the lower panel are results of Southern blotting probed with an internal primer, lanes similar as upper panel. (c) Specific reduction of hTR RNA by hTR-RiAS in Melanoma cancer cell line (A375 SM): Lane 1; 100 bp ladder, lane 2; Sham, and lane 3; mismatch and lane 4; hTR-RiAS. (d) Specific reduction of hTR RNA by hTR-RiAS in Lung cancer cell line (A549): Lane 1; 100 bp ladder, lane 2; sham, lane 3; liposome only, lane 4; scramble, lane 5; mismatch, lane 6; 0.1 μg hTR-RiAS, lane; 7 0.5 μg hTR-RiAS and lane 8; 1.0 μg hTR-RiAS. (e) Specific reduction of hTR RNA by hTR-RiAS in Lung cancer cell line (NCI H1299): Lane 1; 100 bp ladder, lane 2; Sham, and lane 3; liposome alone, lane 4; scramble, lane 5; mismatch and lane 6; hTR-RiAS.

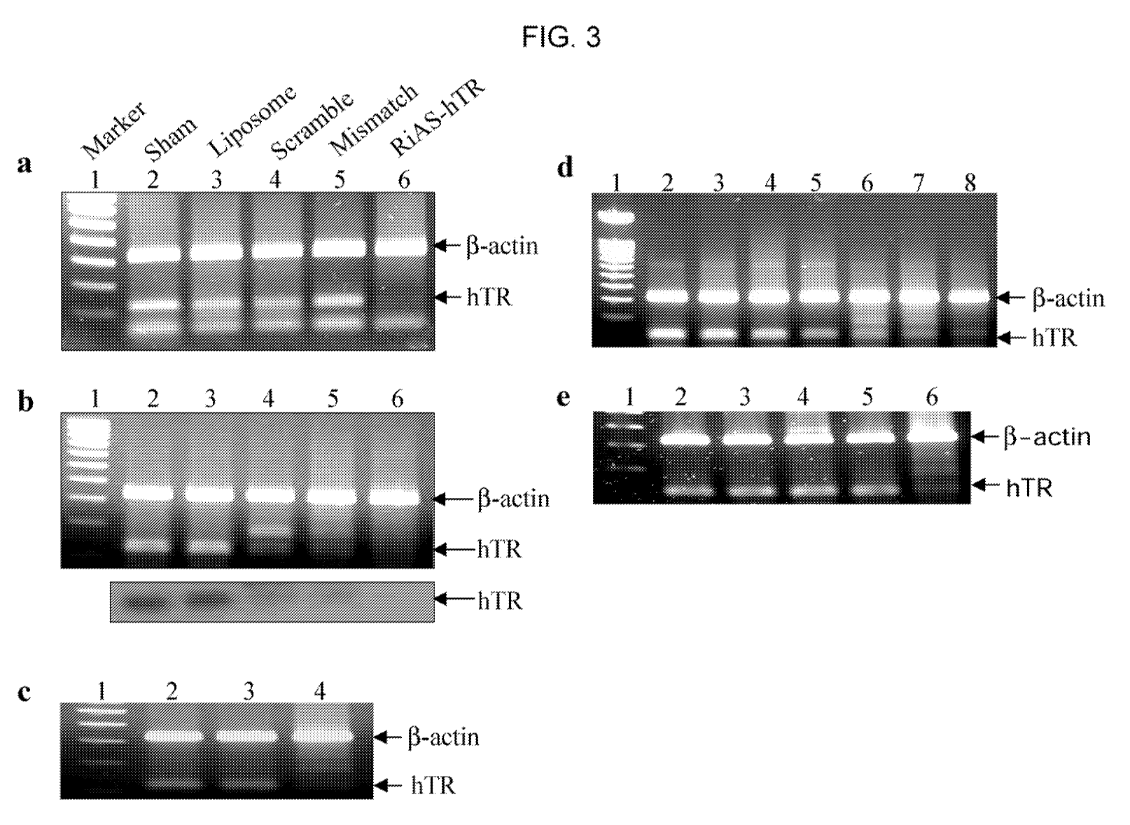

FIGS. 4a-4c show quantification of hTR level by the fluorescence-based real-time reverse transcription polymerase chain reaction (RT-PCR). (a) Amplification graph of hTR level in cell alone, Liposome alone, Mismatch and hTR-RiAS. (b) Amplification graph of β-actin level in cell alone, liposome alone, mismatch, and hTR-RiAS. (c) Quantitative inhibition of hTR level represented by bar graph.

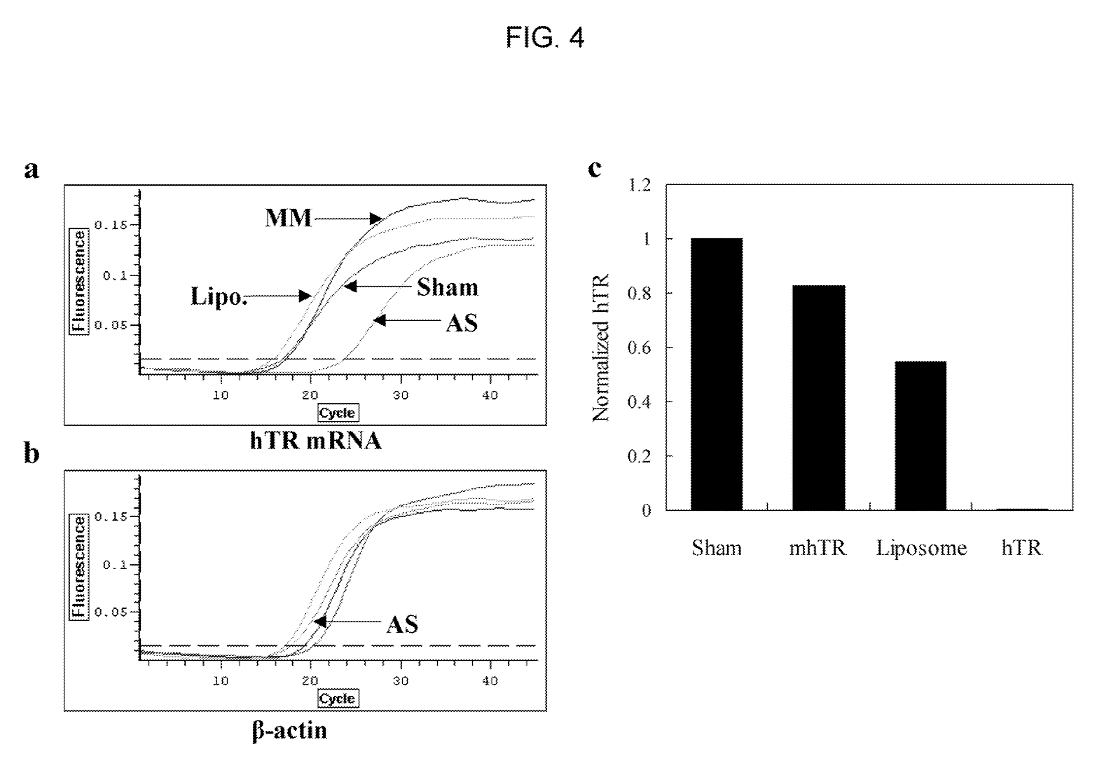

FIG. 5 shows telomerase activity in HeLa cells measured by TRAP-ELISA method. Telomerase activity in hTR-RiAS treated cells was significantly reduced as compared to sham, liposome alone and mismatch.

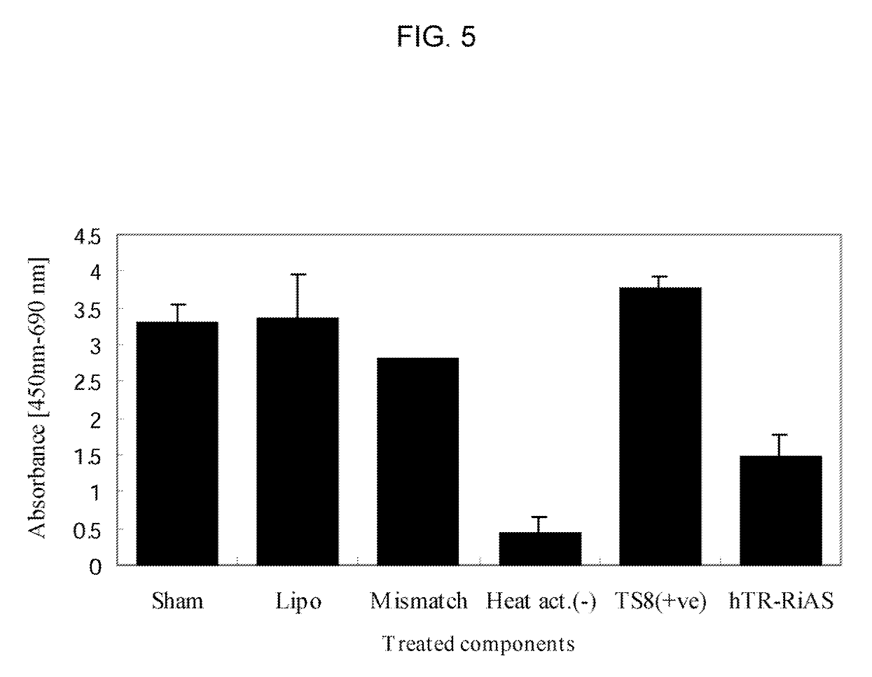

FIGS. 6a-6b show effect of hTR-RiAS oligonucleotides on proliferation of cervical cancer cell line (HeLa) determined by MTT assay. (a) HeLa cells were treated with tripartite lipoplex containing 0.05 μg, 0.1 μg and 0.2 μg of hTR-RiAS. Each bar represents percentage cell growth inhibition of Sham, liposome alone and dose dependant hTR RiAS. Scramble and mismatch were treated in similar ways (data not shown). Each bar value represents the mean±S.D. of triplicate. (b) Growth inhibition of HeLa cells after transfection with different doses of hTR-RiAS oligonucleotides. a; Sham; b; Liposome alone, c; 0.05 μg hTR-RiAS, d; 0.1 μg hTR-RiAS, and d; 0.2 μg of hTR-RiAS.

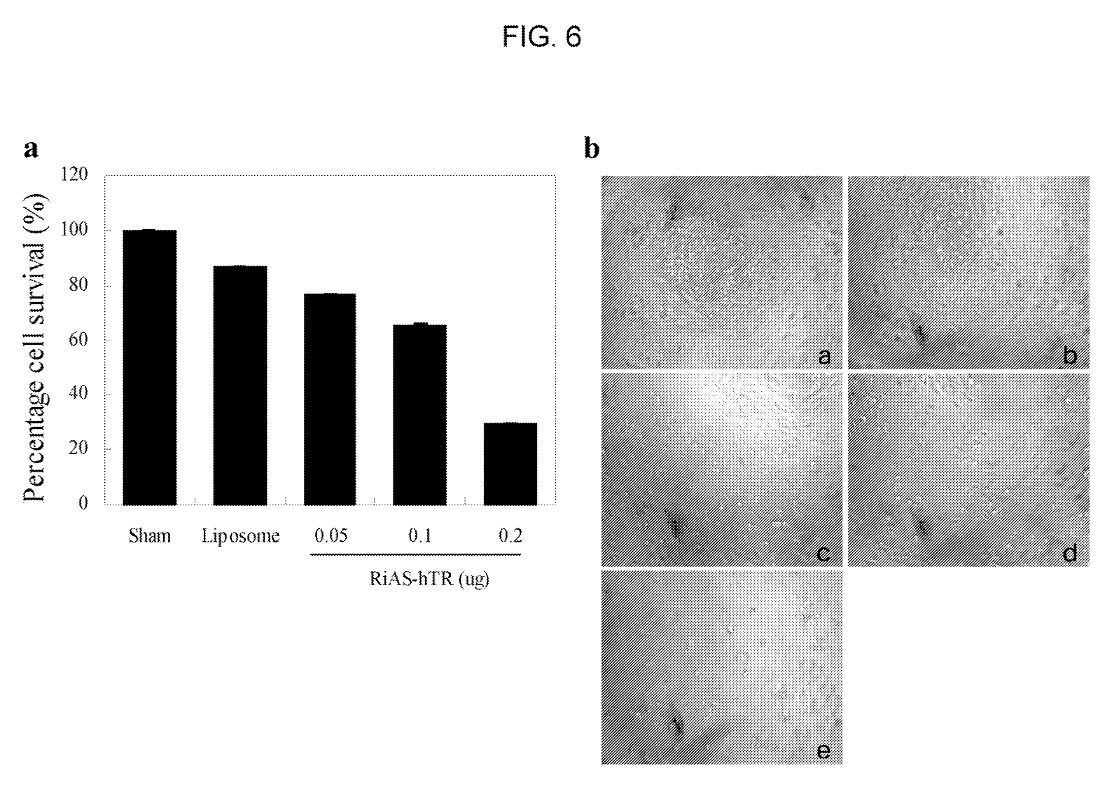

FIGS. 7a-7b show DNA fragmentation assay. Cancer cell lines (a) HeLa cells and (b) Hep3B cells. Cells lysed with a cell lysis buffer and electrophoretically separated in ladder form in 1.8% agarose gel with EtBr staining. Lane 1; 100 bp, ladder, lane 2; Sham, lane 3; liposome alone, lane 4; scramble, lane 5; mismatch, and lane 6; hTR-RiAS.

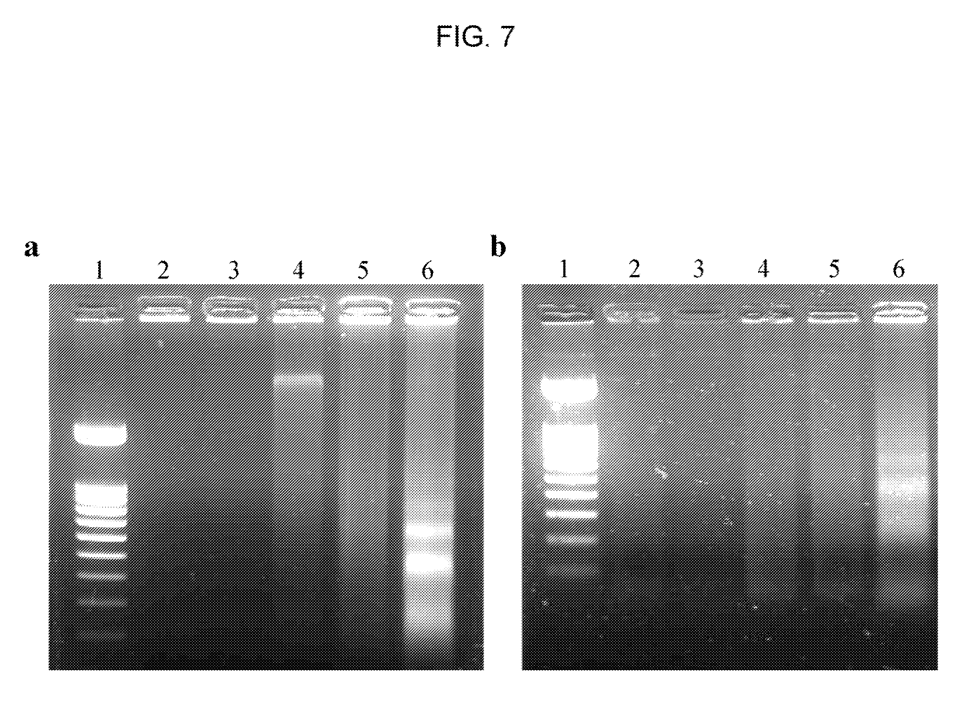

FIG. 8 shows reduction in size of SW 480 subcutaneous tumors in nude mice by hTR-RiAS. hTR-RiAS treated tumors significantly reduced the size of tumor than other control groups.

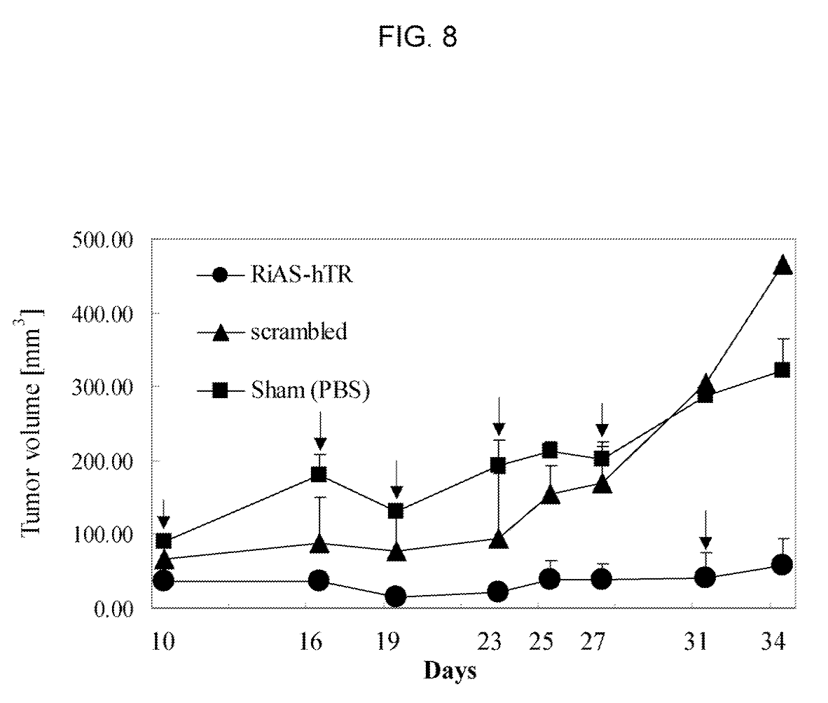

FIGS. 9a-9c show quantitative detection of hTR level by the real-time reverse transcription polymerase chain reaction in vivo (a) Amplification graph of hTR level in Cell alone, Liposome alone, Mismatch and hTR-RiAS. (b) Amplification graph of β-actin level in Cell alone, Liposome alone, Mismatch and hTR-RiAS. (c) Quantitative inhibition of hTR level representing by bar graph.

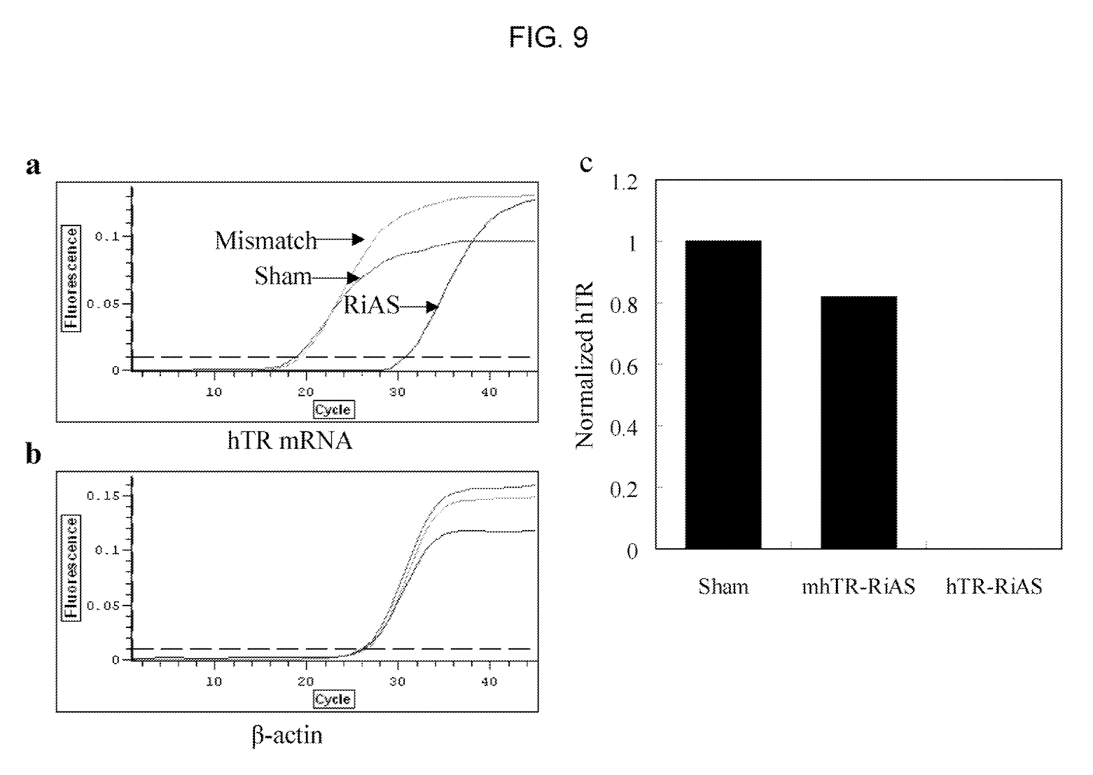

FIG. 10 shows in vivo apoptotic TUNEL assay. Detection of apoptosis by in situ end-labeling of DNA (terminal deoxynucleotidyltransferase (TdT)-mediated dUTP nick end labeling (TUNEL). (a) Cells observed under light microscope. (b) OPTI-MEMI treated tumor cells observed under green fluorescent light. (c) Mismatch treated tumor cells observed under green fluorescent light. (d) hTR-RiAS treated tumor cells observed under green fluorescent light (cells undergone apoptosis are shown in green color). Images (×100).

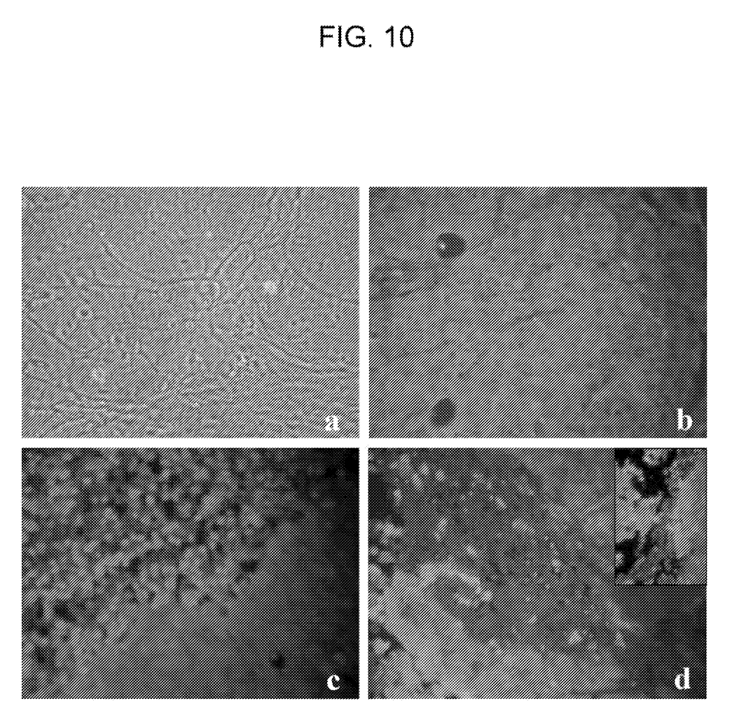

### DETAILED DESCRIPTION OF THE PREFERRED EMBODIMENTS

In the present application, “a” and “an” are used to refer to both single and a plurality of objects.

The present application discloses potent antisense activity of ribbon antisense oligodeoxynucleotides (ODN) to hTR when combined with enhanced uptake of the antisense molecules using the DPL system.

As used herein, the term “antisense” or “AS” means antisense nucleic acid (DNA or RNA) and analogs thereof and refers to a range of chemical species having a range of nucleotide base sequences that recognize polynucleotide target sequences or sequence portions through hydrogen bonding interactions with the nucleotide bases of the target sequences. The target sequences may be single- or double-stranded RNA, or single- or double-stranded DNA.

Such RNA or DNA analogs comprise but are not limited to 2′-O-alkyl sugar modifications, methylphosphonate, phosphorothioate, phosphorodithioate, formacetal, 3′-thioformacetal, sulfone, sulfamate, and nitroxide backbone modifications, amides, and analogs wherein the base moieties have been modified. In addition, analogs of molecules may be polymers in which the sugar moiety has been modified or replaced by another suitable moiety, resulting in polymers which include, but are not limited to, morpholino analogs and peptide nucleic acid (PNA) analogs. Such analogs include various combinations of the above-mentioned modifications involving linkage groups and/or structural modifications of the sugar or base for the purpose of improving RNaseH-mediated destruction of the targeted RNA, binding affinity, nuclease resistance, and or target specificity.

As used herein, “antisense therapy” is a generic term, which includes specific binding of the covalently closed antisense nucleic acid molecules that include an antisense segment for a target gene to inactivate or ablate target RNA sequences in vitro or in vivo.

As used herein, “cell proliferation” refers to cell division. The term “growth,” as used herein, encompasses both increased cell numbers due to faster cell division and due to slower rates of apoptosis, i.e. cell death. Uncontrolled cell proliferation is a marker for a cancerous or abnormal cell type. Normal, non-cancerous cells divide regularly, at a frequency characteristic for the particular type of cell. For instance, when a cell has been transformed into a cancerous state, the cell divides and proliferates uncontrollably. Also, after injury, extracellular cell matrix is overgrown. Inhibition of proliferation or growth modulates the uncontrolled division of the cell or the formation of dense tissue.

As used herein, a “gene” refers to either the complete nucleotide sequence of the gene, or to a sequence portion of the gene.

As used herein, the terms “inhibiting” and “reducing” are used interchangeably to indicate lowering of gene expression or cell proliferation or tissue growth or any other phenotypic characteristic.

As used herein, “substantially complementary” means an antisense sequence having about 70%, 75%, 80%, 85%, 90%, 95%, 96%, 97%, 98%, or 99% homology with an antisense compound which itself is complementary to and specifically binds to the target RNA. As a general matter, absolute complementarity may not be required. Any antisense molecule having sufficient complementarity to form a stable duplex or triplex with the target nucleic acid is considered to be suitable. Since stable duplex formation depends on the sequence and length of the hybridizing antisense molecule and the degree of complementarity between the antisense molecule and the target sequence, the system can tolerate less fidelity in complementarity with larger than conventionally used short linear oligonucleotides of from about 13 to about 30 bases.

As used herein, “substantially similar” means a nucleic acid sequence having about 70%, 75%, 80%, 85%, 90%, 95%, 96%, 97%, 98%, or 99% homology with another nucleic acid. For an antisense molecule having a substantially similar sequence to another antisense molecule directed to the same target, it is the functional capability of the substantially similar molecule that is important, so long as the substantially similar molecule shows target inhibiting activity.

While formation of triplex structure may be within the purview of the present invention, it is understood that such formation is not necessary to practice and obtain the advantageous features of the present invention. For example, it is not necessary to design an oligonucleotide loop structure with parallel and anti-parallel sequences on opposite sides of the loop as disclosed in U.S. Pat. No. 5,683,874.

As used herein, “target” or “targeting” refers to a particular individual gene for which an antisense molecule is made. In certain contexts, “targeting” means binding or causing to be bound the antisense molecule to the endogenously expressed transcript so that target gene expression is eliminated.

The antisense molecule of the invention was found to be superior to conventional linear synthetic AS-oligos in biochemical and biologic activities. While conventional AS-oligos can be easily synthesized by a DNA synthesizer, they require the selection of a target site. The process of selecting for the target site is sometimes termed ‘AS-oligo design’. This process is time consuming and often inconclusive. In addition, synthesized AS-oligos are unstable to nucleases, have frequent sequence errors, entail high production cost, and exhibit poor cellular uptake even after complexation with liposomes.

As used herein, “tat peptide” and “tat-like peptide” are related terms. In particular, tat peptide refers to a portion of the tat protein with possible modifications. Tat-like peptide refers to a peptide that facilitates insertion of nucleic acids into the cell in a similar manner as tat peptide. In one aspect, tat-like peptide may share sequence similarity, in other aspects, the tat peptide may share tertiary or charge similarity with tat peptide, so long as transport of the antisense compound of the invention is facilitated into the cell. Throughout the application, where tat peptide is mentioned as facilitating transport of the antisense molecule into a cell, it can be assumed that tat-like peptide may also be used.

As used herein, “treatment” or “treating” means any treatment of a disease in a mammal, including:
a) preventing the disease, that is, causing the clinical symptoms of the disease not to develop;
b) inhibiting the disease, that is, slowing or arresting the development of clinical symptoms; and/or
c) relieving the disease, that is, causing the regression of clinical symptoms.

Telomerase and hTR

Telomerase is a multi component enzyme complex, composed of the telomerase RNA hTR, catalytic subunit hTERT, and Telomerase associated protein TP1. Since hTR is an essential component of the human telomerase complex, targeting hTR to inhibit telomerase activity has been attempted with different approaches of gene silencing, that is antisense, ribozymes, and more recently siRNA (Glukhov et al; 1998; Norton et al; 1996; Kondo et al; 1998; Barbara et al; 2003). Recent investigations indicated that the inhibition of telomerase activity might change the growth mechanism of cancer cells and suppress tumor growth (Avilion et al; 1996). Previous studies targeting the hTR RNA component of the telomerase complex showed reduced telomerase activity, cell viability and tumor growth, but only up to a certain extent for a limited period of time.

Telomerase activity is detected in 85-90% of human tumors, but not in most somatic cells, indicating a broad involvement of hTR expression in human malignancy (Kim et al; 1994; Kondo et al; 2000;). hTR-RiAS was tested in six human cancer cell lines with telomerase expression. Enhanced uptake of hTR-RiAS resulted in almost complete ablation of hTR RNA, effective inhibition of telomerase activity, and blockade of cancer cell proliferation. Thus, an effective approach for eliminating a component of the telomerase complex may prove to be efficacious for a broad spectrum of human cancer.

RiAS ODN has a covalently closed structure with much enhanced stability as well as natural nucleotide composition. Conventional AS oligos with enhanced stability have adopted chemical modifications that have been blamed for various side effects. It has been reported that conventional antisense oligos require larger amount of antisense oligos ranging from 20 to 200 μg/ml to obtain biological effects that may not entirely be sequence specific. Various control and real time PCR data shown in this report demonstrate that hTR RiAS was effective specifically in a lesser amount. Improved antisense activity shown with the inventive composition can be explained by not only the improved properties of AS-oligos but also enhanced cellular uptake.

In general, antisense oligos show poor cellular uptake due to anionic charges on their polymeric backbone. Cellular uptake of oligonucleotides can be improved when complexed with liposomes (Wheeler et al; 1996). However, non-viral delivery vehicles including liposomes do not provide uptake efficiency that is satisfactory for many types of cells, especially cells of primary culture. Beside, liposomes show rapid clearance in circulation, rendering most liposomes unsuitable for in vivo applications. To improve cellular uptake of antisense oligos, the DPL transfection system has been adopted and shown to have 70-90% cells positive for AS-oligos uptake, resulting in significant improvement of transfection efficiency both in vitro and in vivo.

The inhibitory effect of hTR-RiAS was sequence specific because mismatched and scrambled RiAS ODN failed to exhibit any significant antisense effect. RiAS oligonucleotides would be expected to have normal sequence specificity and susceptibility to RNase H activity because the oligos bear no modified nucleotide. An additional advantage of RiAS ODN is that it is unlikely to introduce undesired mutations in the genomic DNA during DNA replication or repair upon recycling of hydrolyzed nucleotides. It is not known that hTR-RiAS has exerted effects similar to 2′,5′-oligoadenylate on cell viability as a result of RNase L activation, which has been shown to induce apoptosis through decreased stability of RNA (Zhou et al; 1997).

Antisense activity was reported on telomere length and cell proliferation in several different cancer cells. When antisense RNA to hTR was expressed in HeLa cells, the cells showed significant shortening of telomere length after one month of treatment, followed by apoptotic cell death at 23-26 population doublings (Feng et al; 1995; Bisoffi et al; 1998). It was postulated that the cell death was due to shortened telomere length that caused chromosomal instability. An interesting observation was that the cytocidal effect of 2-5 A antisense oligos to hTR exhibited inhibition of telomerase activity, and rapid reduction of cell viability detected in 4-6 days (Mukai et al; 2000; Kondo et al; 1998). The rapid onset of growth inhibition was also demonstrated by ribozyme targeting the hTERT component (Kanazawa et al; 1996). These studies suggest the existence of a “fast track” pathway by which diminution of telomerase can interfere with growth of cancer cells and induce rapid cell death, presumably through apoptosis. The difference may be explained by different levels of inhibition of the telomerase complex. Rapid cell death may be caused by higher degree of telomerase inhibition. Interestingly, application of the inventive compound resulted in even faster inhibition of both telomerase activity and cell proliferation by hTR-RiAS in the cancer cell lines tested. It took only 2-3 days before apoptotic cell death was detected, which may be explained by almost complete ablation of hTR RNA. It should be noted that telomerase activity has been suggested to be associated with increased cellular resistance to apoptosis (Counter et al; 1998; Ren et al; 2001; Zhu et al; 2000; Fu et al; 2000; Holt et al; 1999; Zhang et al; 1998).

Targeting telomerase in an effective manner would have potential utility in the treatment of various human cancers because telomerase is ubiquitously expressed in human tumors. hTR RiAS was shown to be effective in almost complete ablation of hTR RNA, and in partial suppression of telomerase activity and tumor growth. Thus, it would still be interesting to search a way for total blockade of tumor growth by dual targeting of other component of the telomere complex or by combination with conventional therapeutic modalities.

Telomerase enzyme is up regulated in 85-90% of malignant tumors and considered to be an attractive target for anti cancer therapy. The inventive ribbon antisense nucleic acid targeted to the hTR RNA is employed to inhibit telomerase activity and cancer cell growth and to reduce tumor size. The antisense molecule, hTR-RiAS, combined with enhanced cellular uptake is shown to effectively inhibit telomerase activity and cause rapid cell death in various cancer cell lines tested. When cancer cells were treated with hTR-RiAS, the level of hTR RNA was reduced by more than 95%. By contrast, both mismatched and scrambled oligonucleotides failed to reduce the level of hTR RNA in a significant manner. Similarly, whereas telomerase activity was significantly inhibited by hTR-RiAS, only marginal inhibition was detected by control treatments. When checked for cancer cell viability, hTR-RiAS inhibited cell growth up to 90% in 4 days in a very rapid manner. Reduced cell viability was found to be caused by apoptosis as DNA fragmentation was detected after antisense treatment of cancer cells. Further, when subcutaneous tumors of a colon cancer cell line (SW 480) were treated intra-tumorally with hTR-RiAS, tumor growth was markedly suppressed with almost total ablation of hTR RNA in the tumor tissue. Cells in the tumor tissue were also found to undergo apoptosis after hTR-RiAS treatment, detected by in situ TUNEL assay. These results show that hTR-RiAS is a powerful anticancer reagent, with a potential for broad efficacy to diverse malignant tumors.

Covalently Closed Antisense Oligo

Conventional wisdom in the field of antisense therapy has discouraged using long antisense molecules because it was thought that longer AS-oligos tend to be less specific, harder to synthesize and inefficient in cellular uptake. Indeed, chemically modified second generation AS-oligos such as phosphorothioate modified oligos, have reduced sequence specificity as the length of the AS-oligos is extended. Furthermore, synthesis of linear AS-oligos becomes increasingly difficult, and sequence fidelity declines markedly as the length of AS-oligos increases. On the other hand, closed covalent antisense oligonucleotide molecules have shown greater stability even though the molecules are longer and contains additional target sites as compared with short linear oligonucleotides.

The RiAS oligo of the invention may be made by ligating together at least two linear oligonucleotides possessing antisense sequence that targets the same or different gene, or multiple targets within a single linear oligonucleotide. The ligation may be made at the ends, preferably at the 5′ ends which are phosphorylated, where a few bases at the 5′ end are substantially complementary to each other so that hybridization and ligation occur resulting in the formation of a ribbon-type oligonucleotide. The length of the molecule is not limited and in particular may be from about 20 to about 1000 nucleotides, about 20 to 700 nucleotides, about 20 to 600 nucleotides, about 20 to 500 nucleotides, about 20 to 400 nucleotides, about 20 to about 300 nucleotides, preferably about 20 to about 150 nucleotides, or more preferably about 20 to about 120 nucleotides.

In a specific embodiment of the present invention, ribbon-type antisense to hTR mRNA was shown to eliminate the target mRNA in a sequence-specific manner. The results of this study indicates that hTR RiAS oligos may be used as a therapeutic agent for treating various types of cancer and reducing or preventing the growth of various types of tumors. The results in this study demonstrate enhanced properties of the ribbon-type antisense molecule.

Tat and Tat-Like Peptide

In general, antisense oligos show poor cellular uptake due to anionic charges on their polymeric backbone. Cellular uptake of oligonucleotides can be significantly improved when complexed with liposomes (Wheeler et al., Proc. Natl. Acad. Sci. USA 93, 11454-11459(1996)). However, nonviral delivery vehicles including liposomes do not provide uptake efficiency that is satisfactory for many types of cells, especially cells of primary culture. Thus, developing an improved transfection reagent would be beneficial for use in both in vitro cell-line studies and in vivo applications. We devised a simple mixture system comprising antisense oligos, tat-like polypeptide, and liposomes or any other carrier to enhance cellular uptake of RiAS oligos. A short fragment of the tat protein has been shown to have properties of nucleic acid condensation, membrane penetration, and nuclear localization. These properties may be of use in enhancing cellular uptake of nucleotide molecules as well as conjugated proteins (Efthymiadis et al., J. Biol. Chem. 273, 1623-1628(1998); Schwartz et al., Curr. Opin. Mol. Ther. 2, 162-167(2000)). The tat peptide was found to be more effective than comparable short peptides with similar properties such as SV 40 large T antigen peptide (Data to be reported elsewhere).

The specifically exemplified tat peptide in the present application has the amino acid sequence: RKKRRQRRRPPQC (SEQ ID NO:10). However, it is understood that other sequences are included within the purview of the tat peptide of the invention. For instance, RKKRRQRRRPPQ (SEQ ID NO:11) (49-59 of tat protein), may be used. In addition, 86 tat proteins may be used. Modifications to the tat peptide is permissible, such as but not limited to carboxyl group modification of RKKRRQRRRPPQ (e.g.: tat-RGD). Moreover, other sequences may be used as well, such as the first exon (48-72 amino acid) portion of the tat protein.

In another aspect of the invention, other tat-like peptides may be used, such as without limitation, Antp, W/R, NLS, AlkCWK16, DiCWK18, Transportan, K16RGD, VP22, SCWKn, (LARL)n, HA2, RGD, L oligomer, SV40, and the like, so long as the peptides facilitate the insertion of the antisense compound into the cell.

In one embodiment of the invention, the carrier may be covalently linked to the tat or tat-like protein or any other carrier peptide, and may be otherwise complexed or mixed with the tat or tat-like protein or any other carrier peptide that may be used.

Therapeutic Composition

In one embodiment, the present invention relates to treatment for various types of cancer or abnormal cell proliferation caused by abnormal expression or activation of telomerase, where inhibition of expression of the gene is desired. In this way, the inventive therapeutic compound may be administered to human patients who are either suffering from or prone to suffer from the disease by providing compounds that inhibit the expression of telomerase. Types of cancer may include without limitation lung cancer, liver cancer, colon cancer, cervical cancer, or melanoma.

The formulation of therapeutic compounds is generally known in the art and reference can conveniently be made to Remington's Pharmaceutical Sciences, 17th ed., Mack Publishing Co., Easton, Pa., USA. For example, from about 0.05 μg to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intra nasal, intradermal or suppository routes or implanting (eg using slow release molecules by the intraperitoneal route or by using cells e.g. monocytes or dendrite cells sensitized in vitro and adoptively transferred to the recipient). Depending on the route of administration, the peptide may be required to be coated in a material to protect it from the action of enzymes, acids and other natural conditions which may inactivate the ingredients.

For example, the low lipophilicity of the antisense molecules will allow them to be destroyed in the gastrointestinal tract by enzymes capable of cleaving peptide bonds and in the stomach by acid hydrolysis. In order to administer the antisense molecules by other than parenteral administration, they will be coated by, or administered with, a material to prevent its inactivation. For example, antisense molecules may be administered in an adjuvant, co-administered with enzyme inhibitors or in liposomes. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEP) and trasylol. Liposomes include water-in-oil-in-water CGF emulsions as well as conventional liposomes.

The active compounds may also be administered parenterally or intraperitoneally. Dispersions can also be prepared in glycerol liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, theomersal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterile active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from a previously sterile-filtered solution thereof.

When the antisense molecules are suitably protected as described above, the active compound may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings antibacterial and antifungal agents, isotonic and absorption delaying agents and the like. The use of such media and agents for pharmaceutically active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 μg/ml of carrier. In the case of compositions containing supplementary active ingredients, the dosages are determined by reference to the usual dose and manner of administration of the said ingredients.

Administration of the compounds of the invention or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administration are customary in treating the indications that are the subject of the present invention.

Pharmaceutically acceptable compositions include solid, semi-solid, liquid and aerosol dosage forms, such as, e.g., tablets, capsules, powders, liquids, suspensions, suppositories, aerosols or the like. The compounds can also be administered in sustained or controlled release dosage forms, including depot injections, osmotic pumps, pills, transdermal (including electrotransport) patches, and the like, for prolonged and/or timed, pulsed administration at a predetermined rate. Preferably, the compositions are provided in unit dosage forms suitable for single administration of a precise dose.

In addition, the inventive antisense molecules can be co-administered with other active medicinal agents and/or administered in conjunction with other anticancer, antitumor, or anti-proliferative disease therapies. Such therapies include, but are not limited to, radiation therapy, chemotherapy, immunotherapy, laser/microwave and thermotherapy. See Moeller et al., Cancer Cell 2004 5:429-441. Suitable additional active agents include, for example: with alfa interferons such as Interferon alfa-2b; alkylators such as asaley, AZQ, BCNU, busulfan, carboxyphthalatoplatinum, CBDCA, CCNU, CHIP, chlorambucil, chlorozotocin, clomesone, cyclodisone, cyclophosphamide, dacarbazine, dianhydrogalactitol, fluorodopan, hepsulfam, hycanthone, L-PAM, melphalan, methyl CCNU, mitomycin C, mitozolamide, nitrogen mustard, PCNU, piperazine alkylator, piperazinedione, pipobroman, porfiromycin, spirohydantoin mustard, temozolomide, teroxirone, tetraplatin, thio-tepa, triethylenemelamine, uracil nitrogen mustard, and Yoshi-864; anthracyclines such as doxorubicin, cyanomorpholinodoxorubicin, mitoxantrone, idarubicin, doxorubicin liposomal, valrubicin, epirubicin, daunomycin, and daunorubicin liposomal; antibiotics such as dactinomycin, actinomycin D, bleomycin, and daunorubicin; aromatases inhibitor such as anastrozole and letrozole; covalent conjugate of recombinant methionyl human GCSF and monomethoxypolyethylene glycol; cyclo-oxygenase inhibitors such as celecoxib; diluents such as Elliott's B Solution; enzymes such as Asparaginase; erythropoiesis stimulating proteins such as Epoetin alfa and Darbepoetin alfa; estrogen receptor modulators such as tamoxifen and fulvestrant; folate antagonists such as methotrexate; granulocyte colony stimulating factors such as Filgrastim; hormonals such as anastrozole; inorganic arsenates such as arsenic trioxide; microtubule inhibitors such as vincristine, vinblastine, paclitaxel, vinorelbine, and docetaxel; modifiers such as leucovorin and dexrazoxane; monoclonal antibodies such as anti-CD20 (Rituximab, ⁹⁰Y-ibrtumomab tiuexetan, and ¹³¹I-tositumomab), anti-CD22 (Epratuzumab and ⁹⁰Y-epratuzumab), anti-HLA-DR (Remitogen), anti-HER2/NEU (Trastuzumab), anti-CD33 (Gemtuzumab ozogamicin), anti-CD52 (Alemtuzumab), anti-carcinoembryonic antigen (⁹⁰Y-CEA-cide), anti-epithelial cellular-adhesion molecule (Edrecolomab), anti-epidermal growth-factor receptor (Cetuximab, h-R3, and ABX-EGF), anti-VEGF (Bevacizumab), anti-VEGFR2 (IMC-1C11), anti-A33 (huA33), anti-G250/MN (G250), anti-Lewis Y antigen (SGN-15 and Hu3S193), and anti-GD3 (KW-2871); nitrosoureas such as procarbazine, lomustine, CCNU, carmustine, estramustine, and carmustine with Polifeprosan 20 Implant; nucleoside analogues such as mercaptopurine, 6-MP, fluorouracil, 5-FU, thioguanine, 6-TG, cytarabine, floxuridine (intraarterial), fludarabine, pentostatin, cladribine, pentostatin, gemcitabine, capecitabine, gemcitabine, and cytarabine liposomal; osteoclast inhibitors such as pamidronate; platinums such as carboplatin, cisplatin, and oxaliplatin; retinoids such as tretinoin, ATRA, alitretinoin, and bexarotene capsules gel; stem cell stimulators such as Oprelvekin; topoisomerase 1 inhibitors such as topotecan and irinotecan; topoisomerase 2 inhibitors such as etoposide, (VP-16), teniposide, (VM-26), and etoposide phosphate; tyrosine kinase inhibitors such as imatinib mesylate; urate-oxidase enzymes such as Rasburicase; and hydroxyurea.

Covalently Closed Antisense Molecule Delivery Carriers

The antisense delivery carrier of the invention may include a variety of chemical compounds or methods that facilitate the delivery of the antisense compounds of the invention into the cell of interest. A nucleic acid delivery method or carrier used in the invention may include and is not limited to cationic liposomes, PEG-lipid, PEG, poly-L-lysine, poly-D-lysine, dendrimer, Poly (D,L-lactic acid), virosomes, electroporation, magnetofection, naked DNA, lipid-polycation-DNA (LPD), folate-conjugated nanometric particles, cationic nanoparticle (NP) coupled to an integrin alphavbeta3-targeting ligand, (modified) virus coupled with DNA, short amphipathic peptide, a gene-activated matrix (GAM), poly(alpha-(4-aminobutyl)-L-glycolic acid) (PAGA), imidazole-containing polymers, chitosan, gelatin, atelocollagen, poly((D), (L)-lactic-co-glycolic acid) (PLGA), cyclodextrin based polymers, histidine and lysine (HK) polymer, glycotargeted delivery systems, porous polymer microspheres, and the like.

Various delivery systems are known and can be used to administer a compound of the invention, e.g., encapsulation in liposomes, microparticles, microcapsules, recombinant cells capable of expressing the antisense compound, receptor-mediated endocytosis, construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical antisense compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody or a peptide of the invention, care must be taken to use materials to which the protein does not absorb. In another embodiment, the compound or composition can be delivered in a vesicle, in particular a liposome. In yet another embodiment, the compound or composition can be delivered in a controlled release system. In one embodiment, a pump may be used. In another embodiment, polymeric materials can be used. In yet another embodiment, a controlled release system can be placed in proximity of the therapeutic target, i.e., the brain, thus requiring only a fraction of the systemic dose.

A composition is said to be “pharmacologically or physiologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient patient.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

Materials and Methods

### Example 1

### Cell Lines and Cultures

Six various human cancer cell lines that were used are the following: lung cancer cell lines (A549, NCI H1299), liver cancer cell line (Hep3B), colon cancer cell line (SW 480), cervix cancer cell line (HeLa), and melanoma cancer cell line (A375 SM). Three cell lines, HeLa, Hep3B, and A375 SM, were purchased from American Type Culture Collections (Virginia, USA). Remaining three cell lines, A 549, NCI H1299, and SW480, were obtained from Korean cell line Bank (Seoul, Korea). A549 cells were cultured in DMEM, and HeLa, Hep3B, and A375 SM in EMEM. NCI H1299 was cultured in RPMI 1640, and SW480 cells in Leibovitz's L-15. Cell culture media were supplemented with 10% heat inactivated fetal bovine serum and 1% penicillin/streptomycin. All cell culture reagents including FBS were purchased from JBI (Daegu, Korea). Routine cell culture practices were strictly followed to keep proper cell density and features.

### Example 2

### Synthesis of Ribbon Type Antisense Oligonucleotide (RiAS)

Ribbon antisense to hTR (hTR-RiAS) was synthesized using by an Expedite 8909 DNA synthesizer (Applied Biosystem, Foster city, CA, USA). Antisense and control (scrambled and mismatched) oligodeoxynucleotides (ODN) were phosphorylated at the 5′ end. Both antisense to hTR and control ODN are used to form stem-loop structures. The intra-molecular stem is formed by complementary sequences at both 5′ and 3′ ends of each ODN. Two identical ODN molecules were joined by single-stranded sequences that were complementary to each other at the 5′ ends. ODN molecules were mixed and heated for 2 min at 95° C. followed by gradual cooling to room temperature. One unit of T4 DNA ligase was added and incubated for 24 h at 16° C. to generate a ribbon type molecule ligated covalently with dyad-symmetry. The ribbon type ODN consists of two loops and one stem connecting the two loops. The ODNs were purified with the Poros HQ anion exchange column (PerSeptive Biosystems, Framingham, Mass., USA). Anion exchange was performed by gradient elution with 1M NaCl solutions. Purified ODNs were dried by an evaporator, and desalted by reverse phase chromatography. Finally, desalted ODNs were precipitated by ethanol and resuspended in ddH₂O.

### Example 3

### Transfection of hTR-RiAS by the DPL Transfection System

Cellular uptake of hTR-RiAS into cancer cells was enhanced by employing a tripartite DPL (DNA: peptide: liposomes) transfection system that was found to be effective in our laboratory. Antisense oligos were complexed with a short modified peptide of the protein transduction domain (PTD) that was derived from the Tat protein (Efthymiadis et al; 1998), and the complex was added with cationic liposomes (Invitrogen, Carlsbad, Calif., USA) to form a tripartite complex. The ratio of each component of the tripartite DPL (DNA/peptide/liposomes) complex at 1:3:5 (w:w:w) was found to significantly improve cellular uptake of the antisense molecules.

### Example 4

### Reverse Transcriptase-Mediated PCR (RT-PCR)

Total RNA was isolated with the TRI reagent (MRC, Cincinnati, Ohio) according to manufacturer's recommendation. Cells were harvested 24 h after transfection, and subjected to RNA purification using the TRI reagent. The optical density of total RNA was measured at 260 nm with a spectrophotometer. One μg of total RNA was used to perform RT-PCR in a single reaction tube with an Access™ RT-PCR kit (Promega, Madison, Wis.). One microgram of purified total RNA, PCR primers, AMV reverse transcriptase (5 units/μl), Tf1 DNA polymerase (5 units/μl), dNTP (10 nm/1 μl), and MgSO₄ (25 mM, 2.5 μl) were added into a RT-PCR tube. The cDNA amplification was carried out in a PTC-100™ thermal cycler (MJ Research Inc., Watertown, Mass., USA), employing hTR specific primers 5′ gtctaaccctaactgagaag 3′ (SEQ ID NO:12) as a forward primer and 5′ ctagaatgagaagg 3′ (SEQ ID NO:13) as a backward primer. The PCR reactions were carried out once for 45 min at 48° C., 2 min at 94° C., and 35 cycles for 30 sec for denaturation at 94° C., 1 min for annealing at 58° C. and 1 min for extension at 68° C.

RT-PCR products were electrophoretically separated in 1.8% agarose gel with EtBr staining. DNA was transferred onto a nylon membrane (Bio-Rad, Herculeo, Calif., USA) for 6 hrs in 0.4N sodium hydroxide solution. The nylon membrane was then hybridized with an internal primer (5′ ctcgctgactttcagcgggcggaaaag 3′ (SEQ ID NO:14)), the sequence of which was derived from an internal sequence of the amplified hTR fragment. The primer was labeled with 3′-end oligo labeling kit (Amersham Pharmacia Biotech, Buckinghamshire, UK). Hybridization was carried out for 60 min at 60° C. in 6 ml of hybridization buffer containing 5×SSC, 0.2% SDS. The membrane was washed twice in 5×SSC containing 0.02% SDS and twice again with 1×SSC containing 0.1% SDS for 15 min at 58° C. The membrane was blocked with a blocking solution and then treated with anti-fluorescein horseradish peroxidase-conjugated antibody for 30 min before autoradiography.

### Example 5

### Real Time PCR

For quantification of hTR RNA, real-time RT-PCR was performed using DNA Engine 2 OPTICON™ thermal cycler (MJ Research Inc. Waltham, Mass., USA). The hTR level was measured by a continuous fluorescence detector using the SYBR green dye. One microgram of purified total RNA, PCR primers, AMV reverse transcriptase (5 units/μl), dNTP (10 nm/1 μl), MgSO₄ (25 mM, 2.5 μl) and oligo dT primer (1 μl) were added into a RT-PCR tube. The cDNA synthesis was first carried out at 48° C. for 45 min with subsequent inactivation of reverse transcriptase at 94° C. for 2 min. Real time PCR was performed in a total reaction volume of 24 μl that includes 10 μl from previous reaction, 2× cyber buffer (12 μl), forward and backward hTR primers (0.5 μl each), and nuclease free water (1 μl). RT-PCR reactions were first carried out for 10 min at 95° C., and 44 cycles for 30 sec at 94° C., 1 min at 58° C., 2 min at 68° C. Melting curve from 65° C. to 95° C. was read every 0.2° C., held for 1 sec and finally 10 sec at 25° C.

### Example 6

### Cell Viability (MTT) Assay

Percent growth inhibition of different cancer cell lines (SW480, HeLa, A549 and NCI H1299) was determined by MTT assays. Cells were plated at 5×10³ cells/well in a 96-well plate one day prior to transfection and incubated in an atmosphere of 5% CO₂ at 37° C. Next day, cells were washed twice with reduced serum medium i.e. TOM-Transfection Optimized Medium (JBI, Deagu, Republic of Korea), and were transfected with hTR-RiAS ODN in a 50 μl volume for 6 h. Cells were then added with 100 μl of complete medium containing 20% FBS and cultured for 4 days. Cells were harvested in a 50 μl medium and added with 20 μl (5 mg/ml) MTT reagent prepared in phosphate buffered saline, followed by 4 hour incubation in a CO₂ incubator at 37° C. Cells were added with 150 μl dimethyl sulfoxide and incubated one hour at room temperature with gentle mixing. Absorbance was measured at 570 nm by an ELISA reader, Spectra Max 190 (Molecular Device, Sunnyvale, Calif., USA). Percentage growth inhibition was calculated by the following formula: Percent growth inhibition=[1−(Absorbance of an experimental well/absorbance of a sham treated control well)]×100.

### Example 7

### Telomerase Activity (PCR ELISA) Assay

Telomerase activity was determined by using a Telo TAGGG Telomerase PCR ELISAPLUS kit (Roche Diagnostics GmbH, Mannheim Germany), as recommended by the manufacturer. HeLa cells were seeded at 1×10⁶ cells per well in 6 well plates. Cells were transfected with hTR-RiAS and incubated for 48 h. Cells were then trypsinized and lysed in 500 μl of ice-cold lysis buffer for 30 min. The lysates were centrifuged at 13,000 rpm for 30 min at 4° C. and the supernatant was rapidly frozen and stored at −70° C. One micro liter of each extract corresponding to 1×10³ cells were assayed to detect telomerase activity in 50 μl of the total reaction mixture. Telomeric repeats had been added to a biotin-labeled primer during the first telomerase-mediated extension reaction. The elongation products were amplified by PTC-100™ thermal cycler (MJ Research Inc.). An aliquot of the PCR product was denatured, hybridized to a DIG-labeled, telomeric repeat-specific probe and bound to a streptavidin-coated 96-well plate. Finally, the immobilized PCR product was detected with an anti-DIG-POD antibody that was visualized by a color reaction using the substrate TMB, and quantified photometrically at 450 nm.

### Example 8

### DNA Fragmentation Assay

Detection of apoptosis was performed with DNA fragmentation assays. In brief, 1×10⁶ cells were plated in each well of a 6-well plate one day prior to hTR-RiAS treatment. Cells were lysed 24 h after transfection with a lysis buffer containing 1% NP-40, 25 mM EDTA, and 50 mM Tris-HCl. Cell lysate was treated with RNase A (20 μg/ml) for 3 h at 58° C., and followed by proteinase K (20 μg/ml) treatment for 3 h at 37° C. DNA was precipitated by adding 0.1 volume of 3 M ammonium acetate and 2.5 volume of ice-cold ethanol and stored for 12 h at −20° C. DNA was dissolved in TE buffer (pH 8.0) and separated on 1.8% agarose gels.

### Example 9

### In Vivo Studies

For the formation of subcutaneous tumor, SW480 cells (1×10⁷) in 200 μl PBS were injected subcutaneously into the right flank of 6-8 weeks old male BALBc (nu/nu) nude mice (six mice for each treatment). Animals were monitored regularly for tumor occurrence, size and weight. Tumor growth was monitored with a caliper every alternate day. After reaching an adequate tumor size (40-50 mm³), animals were injected intratumorally with either OPTI-MEMI medium alone, hTR-RiAS (100 μg/mouse), or mismatched oligos (100 μg/mouse). The RiAS ODNs were mixed with the PTD peptide in a ratio of 1:2 before injection and administered by daily for 6 days. For histological apoptotic TUNEL assay, mice were sacrificed by cervical dislocation a day after the last treatment, and tumors were removed and fixed in 10% formalin. Tissue sections were cut to 4 μm thickness from specimen embedded with the Paraplast embedding medium (Oxford, St. Louis, Mo., USA). The specimen was stained for an apoptotic TUNEL assay using Apoptosis Detection System (Promega, Madison, Wis.) according to manufacture's instructions.

For in vivo real time RT-PCR, total RNA was purified individually from 3 treated groups (OPTI-MEMI, Mismatch & hTR-RiAS) of tumor tissue using the TRI reagent (MRC, Cincinnati, Ohio). RNA was converted into single stranded cDNA by one step PCR. In vivo real-time PCR was performed using DNA Engine 2 OPTICON™ thermal cycler (MJ Research Inc. Waltham, Mass., USA). For in vivo PCR the protocol and PCR conditions were similar as used in vitro which are mentioned earlier.

Statistical Analysis

All data were made in triplicate, and results were expressed as means±standard deviation (SD). Statistical significance was determined by student's t test.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

### REFERENCES

Allsopp R C, Vaziri H, Patterson C. (1992). Proc Natl Acad sci USA; 89, 10114-10118.
Avilion A A, Piatyszek M A, Gupta J, Shay J W, Bacchetti S, and Greider C W. (1996) Cancer Res.; 1; 56(3); 645-50.
Barbara A. Kosciolek, Kriton Kalantidis, Martin Tabler, and Peter T. Rowley (2003). Molecular cancer therapeutics; 2, 209-216.
Bisoffi M, Chakerian A E, Fore M L, Bryant J E, Hernandez J P, Moyzis R K, Griffith J K. (1998). Eur J cancer; 34, 1242-1249.
Blackburn E H. (1991). Nature; 350, 569-573.
Counter C M, Hahn W C, Wei W, Caddle S D, Beijersbergen R L, Lansdorp P M, Sedivy J M and Weinberg R A. (1998). Proc Natl Acad Sci USA; 8; 95(25), 14723-8.
Efthymiadis A, Lyndall J. Briggs, and David A. Jans. (1998). J Biol Chem; 273, 1623-1628.
Feng J, Funk W D, Wang S S, Weinrich A A A, Chiu C P, Adams R R, Chang E, Allsopp R C, Yu J, Le S, West M D, Harley C B, Andrew W H, Greider C W and Villeponteau. (1995). Science; 269, 1236-1241.
Fu W, Begley J G, Killen M W, and Mattson M P. (1999). J. Biol. Chem; 274, 7264-7221.
Fu W, Killen M, Culmsee C, Dhar S, Pandita T K and Mattson M P (2000). J Mol Neurosci. 14, 3-15.
Glukhov A I, Zimnik O V, Gordeev S A and Severin S E. (1998). Biochem. Biophys. Res. Commun; 248, 368-371.
Hahn W C, Stewart S A, Brooks M W, York S G, Eaton E, Kurachi A, Beijersbergen R L, Knoll J H, Meyerson M and Weinberg R A. (1999). Nature Med; 5, 1164-1170.
Harley C B, Futcher B A and Greidner C W (1990). Nature; 345, 458-460.
Harrington L, McPhail T, Mar V, Zhou W, Oulton R, Bass M B, Arruda I and Robinson M O. (1997). Science; 275, 973-977.
Herbert B, Pitts A E, Baker S I, Hamilton, S E, Wright W E, Shay J W and Corey D R. (1999). Proc. Natl. Acad. Sci. USA; 96, 14276-14281.
Holt S E, Glinsky V V, Ivanova A B and Glinsky G V. (1999). Mol carcinog; 25, 241-248.
Ishikawa T, Kamiyama M, Hisatomi H, Ichikawa Y, Momiyama N, Hamaguchi Y, Hasegawa S, Narita T and Shimada H (1999). Cancer Lett., 141, 187-194.
Kanazawa y; Ohkawa K, Ueda K, Mita E, Takehara T, Sasaki Y, Kashahara A, Hayashi N. (1996). Biochem Biophys Res Commun; 225, 570-576.
Kim N W, Piatyszek M A, Prowse K R, Harley C B, West M D, Ho P L C, Oviello G M, Wright W E, Weinrich S L and Shay J W. (1994). Science; 266, 2011-5.
Kondo S, Kondo Y, Li G, Silverman R H and Cowell J K (1998). Oncogene; 16, 3323-3330.
Kondo Y, Koga S, Komata T and Kondo S. (2000). Oncogene; 19, 2205-2211, 2000.
Matsuda M, Park J G, Hunter S, Chien P, and Schreiber A D. (1996). Mol. Biol. Cell. 7, 1095-1106.
Moon I J, Choi Kyusam, Choi Y K, Kim J E, Lee Y, Schreiber A D and Park J G (2000) (A). J. Biol. Chem; 275 (7-18), 4647-4653.
Moon I J, Lee Y, Kwak C S, Lee J H, Choi Kyusam, Schreiber A D and Park J G. (2000) (B) Biochem. J; 346, 295-303.
Mukai S, Kondo Y, Koga S, Komata T, Bama B P and Kondo S (2000). Cancer Res; 60, 4461-4467.
Meyerson M, Counter C M, Eaton E N, Ellisen L W, Steiner P, Caddle S D, Ziaugra L, Beijersbergen R L, Davidoff M J, Liu Q, Bacchetti S, Haber D A and Weinberg R A (1997). Cell; 90, 785-795.
Naka K, Yokozaki H, Yasui W, Tahara H, Tahara E, Tahara E. (1999). Biochem. Biophys. Res. Commun; 255, 753-758.
Nakamura T M, Morin G B, Chapman K B, Weinrich S L, Andrews W H, Lingner J, Harley C B and Cech T R. (1997). Science; 277, 955-959.
Nakayama J, Saito M, Nakamura H, Matsuura A and Ishikawa F. (1997). Cell; 88, 875-884.
Norton J C, Piatyszek M A, Wright W E, Shay J W and Corey D R. (1996). Nat. Biotechnol; 5, 615-619.
Nugent C I, Bosco G, Ross L O, Evans S K, Salinger A P, Moore J K, Haber J E and Lundblad V. (1998). Curr. Biol; 11, 657-660.
Ren J G, Xia H L, Tian Y M, Just T, Cai G P, and Dai Y R. (2001). FEBS Lett; 19, 488: 133-138.
Shay J W and Wright W E. (1996). Curr Opin Oncol; 8, 66-71.
Urquidi V, Train D, and Goodison S. (2000). Ann. Rev. Med; 51, 65-79.
Wheeler C J, Felgner P L, Tsai Y J, Marshall J, Sukhu L, Doh S G, Hartikka J, Nietupski J, Manthorpe M, Nichols M, Plewe M, Liang X, Norman J, Smith A and Cheng S H. (1996). Proc natl Acad Sci USA; 93, 11454-11459.
Zhang X, Vernon Mar, Wen Zhou, Lea Harrington, and Murray O. Robinson. (1998). Genes Dev; 13, 2388-2399.
Zhou A, Paranjape J, Brown T L, Nie H, Naik S, Dong B, Chang A, Trapp B, Fairchild R, Colmenares C and Silverman R H (1997). EMBO J; 16, 6355-6363.
Zhu H, Weiming Fu, and Mark P. Mattson. (2000). J Neurochem; 75, 117-124.

1. A purified covalently closed antisense molecule, which specifically inhibits expression of human telomerase by specifically binding to nucleic acid encoding human telomerase.

2. The covalently closed antisense molecule according to claim 1, wherein the molecule has at least two loops separated by a stem structure, wherein at least one loop comprises a target antisense sequence that specifically binds to nucleic acid encoding human telomerase.

3. The covalently closed antisense molecule according to claim 2, wherein the molecule specifically binds to nucleic acid encoding hTR.

4. The covalently closed antisense molecule according to claim 3, which comprises a sequence, which is substantially similar to SEQ ID NO:1, SEQ ID NO:2, SEQ ID NO:3, SEQ ID NO:4, or SEQ ID NO:5.

5. A method of making the molecule according to claim 2, comprising ligating together at least two linear antisense molecules with stem-loop structure having either or both 5′ or 3′ ends be substantially complementary to each other so that a covalently closed antisense molecule is made.

6. The method according to claim 5, wherein the linear antisense molecule may be specific for the same target nucleic acid or a different nucleic acid.

7. A method of inhibiting expression of telomerase comprising contacting a sample comprising telomerase expressing cells with the covalently closed antisense molecule according to claim 1.

8. A method of treating a condition caused by expression of telomerase, comprising administering the covalently closed antisense molecule according to claim 1 to a subject in need thereof.

9. The method according to claim 8, wherein said condition is cancer.

10. The method according to claim 9, wherein the cancer is carcinoma.

11. The method according to claim 9, wherein the cancer is lung cancer, liver cancer, colon cancer, cervical cancer, or melanoma cancer.

12. A method for preventing proliferation of cells or reducing tumor growth or size, comprising administering a composition comprising the covalently closed antisense molecule according to claim 1 to a subject in need thereof.

13. The method according to claim 12, wherein the tumor is carcinoma.

14. The method according to claim 12, wherein the tumor is lung tumor, liver tumor, colon tumor, cervical tumor, or melanoma tumor.

15. A composition comprising the covalently closed antisense molecule according to claim 1, tat or tat-like peptide, and a carrier composition.

16. The composition according to claim 15, wherein the carrier is a liposome.

17. The composition according to claim 15, wherein the covalently closed antisense molecule is targeted to hTR.

18. A method of delivering a covalently closed antisense molecule according to claim 1 to a cell, comprising contacting the cell with the covalently closed antisense molecule, a tat or tat-like peptide and a carrier composition.

19. The method according to claim 18, wherein the tat or tat-like peptide and the carrier composition are mixed before contacting the cell.

20. A method for treating cancer comprising administering a combination of component (i) a composition comprising the molecule according to claim 1; and component (ii) radiation therapy, immunotherapy or chemotherapy to a subject in need thereof, with sufficient dosage or amount of the combination of component (i) and component (ii) to be effective for treating cancer or the symptoms of cancer.


SEQUENCE LISTING (14 sequences)

SEQ ID NO: 1 - DNA, 36 bp, Artificial sequence
  Oligo
acattttttgtttgctctagaatgaacggtggaagg

SEQ ID NO: 2 - DNA, 40 bp, Artificial sequence
  Oligo
aaaatggccaccacccctcccaggcccaccctccgcaacc

SEQ ID NO: 3 - DNA, 40 bp, Artificial sequence
  Oligo
aaagtcagcgagaaaaacagcgcgcggggagcaaaagcac

SEQ ID NO: 4 - DNA, 31 bp, Artificial sequence
  Oligo
aaaacagagcccaactcttcgcggtggcaaa

SEQ ID NO: 5 - DNA, 31 bp, Artificial sequence
  Oligo
aaacgggcgagtcggcttataaagggagaaa

SEQ ID NO: 6 - DNA, 36 bp, Artificial sequence
  Oligo
atatttcttgtctgctcttgattgaaccgttgaatg

SEQ ID NO: 7 - DNA, 36 bp, Artificial Sequence
  Oligo
tcagtctagttagttaggtctatgatgactgagtag

SEQ ID NO: 8 - DNA, 50 bp, Artificial sequence
  Oligo
gatcgtcccggaaggtggcaagtaagatctcgtttgttttttacagggac

SEQ ID NO: 9 - DNA, 50 bp, Artificial sequence
  Oligo
gatccagggacattttttgtttgctctagaatgaacggtggaaggccctg

SEQ ID NO: 10 - protein, 13 aa, Artificial sequence
  Peptide
RKKRRQRRRPPQC

SEQ ID NO: 11 - protein, 12 aa, Artificial sequence
  Peptide
RKKRRQRRRPPQ

SEQ ID NO: 12 - DNA, 20 bp, Artificial sequence
  Primer
gtctaaccctaactgagaag

SEQ ID NO: 13 - DNA, 14 bp, Artificial sequence
  Primer
ctagaatgagaagg

SEQ ID NO: 14 - DNA, 27 bp, Artificial sequence
  Primer
ctcgctgactttcagcgggcggaaaag